(19) United States
(12) Patent Application Publication
(10) Pub. No.: US 2018/0338715 A1
(43) Pub. Date: Nov. 29, 2018

(54) TECHNOLOGY AND METHODS FOR DETECTING COGNITIVE DECLINE

(71) Applicant: Newton Howard, Providence, RI (US)

(72) Inventor: Newton Howard, Providence, RI (US)

(21) Appl. No.: 15/988,260

(22) Filed: May 24, 2018

Related U.S. Application Data
(60) Provisional application No. 62/510,498, filed on May 24, 2017.

(51) Int. Cl. A61B 5/00; A61B 5/11

(52) U.S. Cl. CPC A61B 5/4088; A61B 5/1118; A61B 5/6887; A61B 5/002; A61B 5/1113

(57) ABSTRACT

Embodiments of the present systems and methods may detect cognitive decline using changes in activities of daily living. For example, in an embodiment, a computer-implemented method for determining effects of cognitive loading on a person may comprise receiving data from at least one physical movement sensor attached to a person, the data recorded while the person is repeatedly performing a physical task and while the person is under cognitive loading during at least some of the performances of the task, determining physical movements of the person in response to the cognitive loading from the received data for each of the performances of the task, and determining an effect of cognitive loading based on the physical movements of the person while performing the physical task without cognitive loading and while performing the physical task with cognitive loading.

### CROSS-REFERENCE TO RELATED APPLICATIONS

The application claims the benefit of U.S. Provisional App. No. 62/510,498, filed May 24, 2017, which is incorporated herein in its entirety.

### BACKGROUND

The present invention relates to techniques for performing Bayesian assessment of real-world behavior during multitasking.

Cognitive decline due to ageing or disease is a common occurrence. Generally, early detection of cognitive decline may provide the opportunity for early treatment, along with slowing the progression of such decline. Multitasking is common in everyday life, but its effect on activities of daily living is not well understood. Critical appraisal of performance for both healthy individuals and patients is required. Cognitive decline due to ageing or disease may affect the performance of activities of daily living. Likewise, detection of such changes in the performance of activities of daily living may indicate cognitive decline. Accordingly, a need arises for techniques that may detect cognitive decline using changes in activities of daily living.

### SUMMARY

Embodiments of the present systems and methods may detect cognitive decline using changes in activities of daily living. Motor activities during activities of daily living may be monitored with a wearable sensor network during single and multitask conditions. Motor performance may be quantified by the median frequencies (fₘ) of hand trajectories and wrist accelerations. The probability that multitasking occurred based on the obtained motor information may be estimated using a Naïve Bayes Model, with a specific focus on the single and triple loading conditions. The Bayesian probability estimator may task distinction for the wrist accelerometer data at the high and low value ranges. The likelihood of encountering a certain motor performance during well-established everyday activities, such as preparing a simple meal, may change when additional (cognitive) tasks were performed. Within a healthy population, the probability of lower acceleration frequency patterns increases when people are asked to multitask. Cognitive decline due to ageing or disease may yield even greater differences.

For example, in an embodiment, a computer-implemented method for determining effects of cognitive loading on a person may comprise receiving data from at least one physical movement sensor attached to a person, the data recorded while the person is repeatedly performing a physical task and while the person is under cognitive loading during at least some of the performances of the task, determining physical movements of the person in response to the cognitive loading from the received data for each of the performances of the task, and determining an effect of cognitive loading based on the physical movements of the person while performing the physical task without cognitive loading and while performing the physical task with cognitive loading.

In embodiments, the physical task may be a task of everyday living. The cognitive loading may be a Stroop task. Determining physical movements of the person may comprise determining a power spectrum of the sensor data, comparing a frequency at which the power spectrum has a maximum amplitude with an expected range of frequencies, and determining that a physical movement has occurred when the frequency at which the power spectrum has a maximum amplitude is within the expected range of frequencies. Determining physical movements of the person may further comprise applying a continuous wavelet transform to the sensor data of a physical movement for which it was determined that the physical movement occurred, generating a scalogram of wavelet coefficients of the continuous wavelet transform, determining that a physical movement began based on the scalogram, determining the response time based on the time a stimulus was given and the determined time that the physical movement began, and identifying the physical movement as correct when the determined physical movement matches an expected physical movement. Determining an effect of cognitive loading may comprise determining differences in response times between the physical tasks performed without cognitive loading and the physical tasks performed with cognitive loading. Determining differences in response times may comprise using a Naïve Bayes probability estimator to distinguish between the physical tasks performed without cognitive loading and the physical tasks performed with cognitive loading.

In an embodiment, a system for identifying determining effects of cognitive loading on a person may comprise at least one physical movement sensor attached to a person and adapted to transmit data representing physical movements of at least a portion of the person and a computing system comprising a processor, memory accessible by the processor, and computer program instructions stored in the memory and executable by the processor, computing system adapted to perform receiving data from the at least one physical movement sensor attached to the person, the data recorded while the person is repeatedly performing a physical task and while the person is under cognitive loading during at least some of the performances of the task, determining physical movements of the person in response to the cognitive loading from the received data for each of the performances of the task, and determining an effect of cognitive loading based on the physical movements of the person while performing the physical task without cognitive loading and while performing the physical task with cognitive loading.

In an embodiment, a computer program product for determining effects of cognitive loading on a person may comprise a non-transitory computer readable storage having program instructions embodied therewith, the program instructions executable by a computer, to cause the computer to perform a method comprising receiving data from at least one physical movement sensor attached to a person, the data recorded while the person is repeatedly performing a physical task and while the person is under cognitive loading during at least some of the performances of the task, determining physical movements of the person in response to the cognitive loading from the received data for each of the performances of the task, and determining an effect of cognitive loading based on the physical movements of the person while performing the physical task without cognitive loading and while performing the physical task with cognitive loading.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary block diagram of a body sensor network in which techniques of the present systems and methods may be implemented.

FIG. 2 is an exemplary flow diagram of an embodiment of a process for determining the extent that a task may be influenced by multitasking.

FIG. 3 is an exemplary illustration of Stroop task response detection.

FIG. 4 is an exemplary illustration of Q-Q plots that may be used to visually check for normality.

FIG. 5 is an exemplary illustration of boxplots that may be used to visualize the hand trajectories and accelerations between conditions.

FIG. 6 is an exemplary illustration of visualisations of the estimated probability distribution between single and triple tasks.

FIG. 7 is an exemplary block diagram of a computer system in which processes involved in the embodiments described herein may be implemented.

### DETAILED DESCRIPTION

Embodiments of the present systems and methods may detect cognitive decline using changes in activities of daily living. Motor activities during activities of daily living may be monitored with a wearable sensor network during single and multitask conditions. Motor performance may be quantified by the median frequencies (fₘ) of hand trajectories and wrist accelerations. The probability that multitasking occurred based on the obtained motor information may be estimated using a Naïve Bayes Model, with a specific focus on the single and triple loading conditions. The Bayesian probability estimator may task distinction for the wrist accelerometer data at the high and low value ranges. The likelihood of encountering a certain motor performance during well-established everyday activities, such as preparing a simple meal, may change when additional (cognitive) tasks were performed. Within a healthy population, the probability of lower acceleration frequency patterns increases when people are asked to multitask. Cognitive decline due to ageing or disease may yield even greater differences.

Much can be learned about the brain by studying motor coordination. Motor behavior, defined as the combination of movements that produce purposeful or intended actions, emerges due to a synergy between a range of systems. The systems involved in this behavior are bounded by certain parameters and they have evolved to work within real-world constrains. Everyday living activities arise through the complex interaction of these factors and dysfunction within these factors will generate alternative behaviors. The occurrence of large changes in everyday living behavior can be an indicator that (patho)physiological changes are emerging. This is also the reason that at present the diagnosis of disorders such as Alzheimer's disease still heavily depends on the clinical history and the observed behavioral changes by relatives and friends. Furthermore, there is growing evidence that indicates a link exists between activities of daily living (ADL) and executive dysfunction in patients suffering from early dementia.

It is unclear how changes in certain parameters might affect the behavior under real-world conditions. The complex interactions underlying behavior can be better understood by, for example, exploring effects of cognitive loading in healthy populations. This information may be particularly interesting if it represents behavior that is common in the real-world. Accordingly, embodiments of the present systems and methods may perform detailed assessments of multitasking within an ADL context, in which the tasks cover a range of complex everyday tasks. Embodiments may determine the extent to which everyday living is affected by cognitive ability.

In the case that complex ADL motor performance is affected by cognitive loading, cognitive loading may be predicted by monitoring motor behavior itself. In embodiments, motor performance is may be determined with variable levels of cognitive loading being introduced. The probability that multitasking occurred based on motor performance data may be estimated with a Naïve Bayes Model. The model is a simple probabilistic method based on Bayes Theorem. In practice Naïve Bayes models often perform rather well compared to more sophisticated models. The simplicity, large community of users and ease of implementation makes the Naïve Bayes Model an ideal candidate for initial exploration of real-world multitasking.

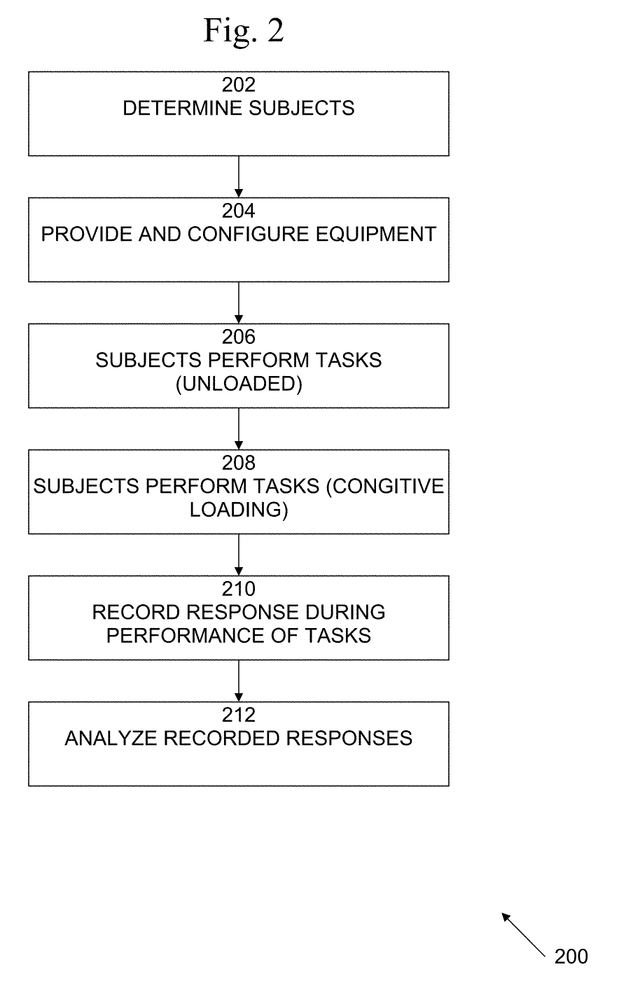

An example of a process for determining the extent that a task may be influenced by multitasking is shown in FIG. 2. Multitasking may increase the probability of observing “slower” motion patterns, as defined by a decrease in the median frequency. Process 200 begins with 202, in which the subjects for task influence determination may be determined. For example, a number of subjects may be determined, which may include male and female subjects, healthy subjects and subjects with medical conditions, subjects of varying ages, etc. In an example, a total of 21 (8 male, 13 female) healthy subjects may be recruited. In this example, the subjects may have a mean age of 23 (±3) years, an average height of 170 (±8) cm and an average weight of 67 (±12) kg. All subjects may give written and informed consent to participate in process 200.

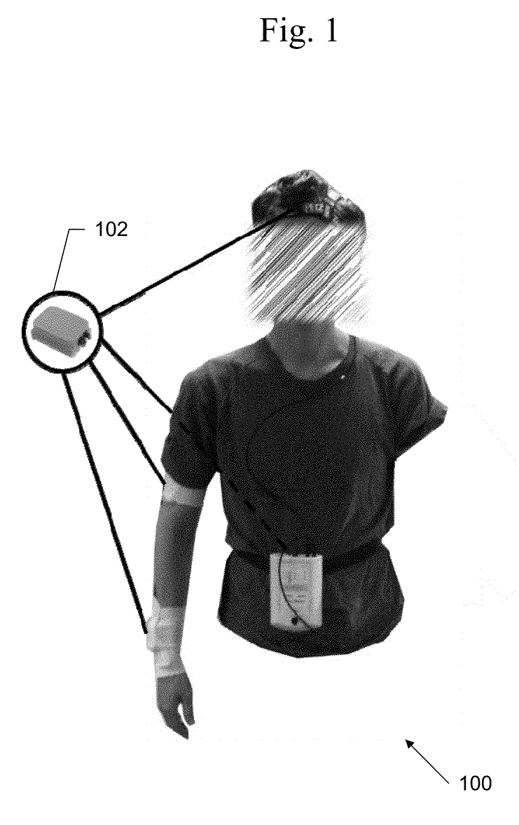

At 204, the equipment to be used may be provided and configured. For example, subjects may wear a body sensor network, such as body sensor network 102, shown in FIG. 1. In this example, the network may include 4 sensors, attached to the right upper arm, right lower arm, head and back. The back sensor may be used as reference sensor to determine if all other sensors worked appropriately. Sensors on the arm and back may be kept in place by double sided tape and straps. The head sensor may be placed on a non-slip elastic headband.

At 206, the subjects may perform a task, such as a task of everyday living, for an indefinite or for a predetermined time period, without any additional cognitive loading. For example, subjects may perform the task of preparing a meal during a 40 second trial. The meal preparation may include making as many sandwiches as possible and may include several tasks. For example, participants may butter and cut as many slices of bread as possible within 40 seconds. Further examples of tasks may be found in, for example, the Motor Activity Log (MAL) for the upper extremity (Uswatte G, Taub E, Morris D, Vignolo M, McCulloch K. Reliability and Validity of the Upper-Extremity Motor Activity Log-14 for Measuring Real-World Arm Use. Stroke. 2005; 36(11):2493-6.).

At 208, the subjects may perform a task, such as a task of everyday living, for an indefinite or for a predetermined time period, with cognitive loading. For example, subjects may be instructed to speak freely and/or perform an additional cognitive activity, such as a Stroop task, while always performing the motor task of 206. In embodiments, four conditions may be implemented, including 1) performing the motor task alone (Single task condition), 2) performing the motor task while speaking (Dual task condition with speech), 3) performing the motor task while conducting a cognitive activity, such as a Stroop task (Dual task condition with Stroop task), and 4) performing the motor activity concurrently with both speaking and a cognition task (Triple task condition). In embodiments, trials of the single task condition may be performed at the start of the process and at the end of the process. Multiple trials may be performed other conditions. The conditions were randomized or pseudo-randomized in order to eliminate sequence effects in the outcomes.

The cognitive loading task may include, for example, a specific audio-spatial assignment. For example, the auditory spatial task may utilize a spatial Stroop stimulus and may be presented through, for example, a wireless stereo headphone. In embodiments, a plurality of stimuli may be presented. For example, within one trial three stimuli may be given, with 10 seconds between each stimulus. The subjects may respond to a unilateral aural stimulus. For example, the stimuli may include the words “Left” and “Right” delivered through either the left or right headphone speaker. For example, if the word matches the side it was presented to, such as “Left” in the left ear, the result is congruous and therefore the appropriate response may be for the subject to indicate it was correct by nodding the head up and down. If the word does not match the side it was presented to, such as “Left” in the right ear, the result is incongruous, the subject may indicate it was incorrect by shaking the head from side to side.

Examples of systems that may be used to generate stimuli generation for the Stroop task, as well to perform data acquisition and analysis may include MATLAB® R2014a (MathWorks Inc., Natick, Mass., USA).

At 210, the response of subjects to the stimuli during performance of the tasks may be recorded. For example, a head-mounted sensor may be used to collect the angular velocities (°/s) in pitch direction (ω_{patch}; indicating “correct”) and yaw direction (ω_{yaw}; indicating “incorrect”). At 212, the recorded responses may be analyzed. For example, the power spectral density P(f) may be estimated for each direction (ω). For example, a method based on applying a Discrete Fourier Transform (DFT), shown below, may estimate the power spectra. The data may then be split into windows, modified periodograms of these windows may be determined, and the obtained periodograms may be averaged. (Welch P. The use of fast Fourier transforms for the estimation of power spectra: A method based on time averaging over short, modified periodograms. IEEE Transactions on Audio and Electroacoustics. 1967; 15(2):70-3.)

$${Ω}_{k + 1} = {\sum }_{j = 0}^{n - 1} {( {e}^{- 2 π i / n} )}^{jk} {ω}_{j + 1} \qquad ( 1 )$$

The DFT equation (Eq. 1) may take in one of the head movement directions (ω_{pitch} or ω_{yaw}) containing n sampled data points, with an index (j). Here, j is the imaginary unit and k the index to output Ω. In embodiments, a Fast Fourier Transform (FFT) may be applied as a more efficient way of computing the required DFT. The frequency at which the power spectral density then reaches its maximum (fₘₐₓᵢₘᵤₘ) may be compared against an expected relevant physiological range of 0.5-10 Hz. Frequencies outside this range may be assumed as unlikely voluntary physiological responses and may be labelled as “no response given”. All signals may be checked for a potential second peak whenever the initially detected peak fell outside the physiological range. This approach may be taken in order to prevent incorrect dismissal of data.

The continuous wavelet transform may be computed for all signals that showed fₘₐₓᵢₘᵤₘ within the selected range. It may be assumed that the nodding response would be best represented by a Morlet wavelet. This wavelet is the product of a complex exponential wave and a Gaussian envelope. The Morlet wavelet's function ψ(t) may be described by:

$$ψ ( t ) = {e}^{\frac{- {β}^{2} {t}^{2}}{2}} cos ( π t ) \qquad ( 2 )$$

in which t is time with β controlling the shape by balancing the time and frequency resolution. The Morlet wavelet may be defined as a “mother” wavelet from which a range of wavelets may be generated by scaling and translating,

$${ψ}_{a , b} ( t ) = \frac{1}{\sqrt{a}} \cdot ψ ( \frac{t - b}{a} ) for a > 0 , b ϵ ℝ \qquad ( 3 )$$

in which a is the scaling parameter and b is the translation parameter, with t denoting the independent variable. The collection of wavelets that arise from this may be used as an orthonormal basis. The relevant coefficients may be obtained by

C_{a,b,f(t),ψ}=∫_{-∞}^{∞}ƒ(t)·ψ_{a,b}(t)dt (4)

Varying the values of a and b may provide the continuous wavelet transform coefficients C_{a,b} indicating how closely the wavelet is correlated to the original signal. These coefficients are of course dependent on the selected waveform (ψ) and function (ƒ). A larger value for C_{a,b} shows a greater similarity between ψ and ƒ.

A scalogram of wavelet coefficients may then be generated. The start of a specific response may be defined as the point when the energy level of the fₘₐₓ scale crossed a pre-set boundary. A limitation with applying a single value crossing is the selection bias. In order to overcome this as much as possible a range of thresholds were explored by

$${T}_{current} = \frac{{E}_{max}}{T} \{ T ∈ ℕ \vert 1 \leq T \leq 100 \} \qquad ( 5 )$$

with Eₘₐₓ being the maximum energy and T the threshold denominator set to produce a current threshold (T_{current}). Analysis of pilot data may indicate that large shifts may be minimized when a T of 22 is applied. To allow for some random variation T may be set to 30. This may give the following formula to detect within a 10 seconds interval the first energy (E) crossing by

$$E > \frac{{E}_{max}}{30} . \qquad ( 6 )$$

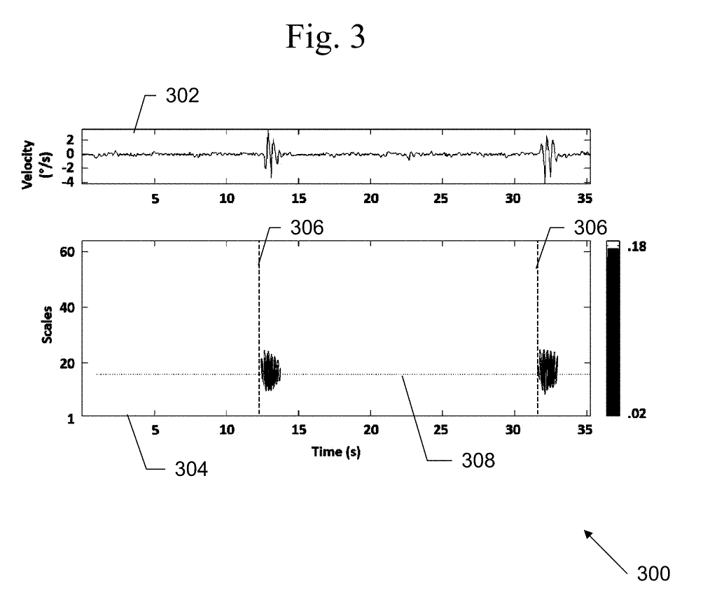

This may show good identification of responses across several pilot test sessions. An example is shown in FIG. 3. FIG. 3 illustrates an example of Stroop task response detection 300 based on energy percentage of each wavelet coefficient. The upper diagram 302 shows the original angular velocity signal in yaw direction across time. The lower diagram 304 shows the scalogram of wavelet coefficients. It provides the percentage of energy for each coefficient depicted by a heat map that is given on the side. Lines 306 show identified crossings of the set threshold 308.

The time at which a certain stimulus was given may be subtracted from the time when a response is detected. This value may represent the response time of the subject. A window size of 10 seconds may be used to identify any responses, as the stimuli were generated at a 0.1 Hz rate. The response may be labelled “incorrect” if no response was found. Identified responses may be compared to the expected response. If the response is expected to occur within a specific direction (yaw or pitch) the response may be labelled “correct”. Otherwise the response may be deemed “incorrect”.

However, it could be that there is a response signal present in both yaw and pitch directions. In this case it needs to be determined if a corrective action (yaw and pitch response are separated in time) has taken place or if it is crosstalk of the channels due to for example vigorous shaking. Crosstalk may be defined as one signal overlapping the other and may be formalized as:

t_{yaw(1)}<t_{pitch(n)}∧t_{pitch(1)}<t_{yaw(n)} (7)

In Eq. 7, t_{yaw}(1) and t_{pitch}(1) are the time points at the start of the response and t_{yaw}(n) and t_{pitch}(n) indicated the end of the response. If any overlap is detected, the signal with the highest average energy may be identified as the leading signal (1 may be assigned) and the other signal is seen as the crosstalk signal (it may be assigned a value of 0.5). If both signals are equal in terms of average energy, they may both be assigned a value of 0.5 and it may be determined that it is inconclusive which response the subject wanted to give.

A truth matrix consisting of dichotomized outcomes may allow for easy assessment of performance. The first two cells of each row may be summed and if this value is greater than 1 the performance may be labelled as correct. This simple computation may provide a quick top-level view of the provided responses. The summed outcomes may be labelled as extracted responses.

Upper limb motion patterns may be obtained through a simple biomechanical model. for example, the Euclidian norm of the hand trajectory may be computed by

∥p∥=\sqrt{pₓ²+p_{y}²+p_{z}²} (8)

with f being the frequency in Hz, fₘₐₓ the maximum frequency in the spectrum, and P(f) the power spectral density. Median frequency may be computed for both ∥p∥ and ∥a∥ for, for example, a 3 second block that was taken directly after the Stroop task stimulus was applied. For the unloaded condition, for example, a 3 second data block may be taken at similar time intervals. All three fₘ within a trial may be used to compute an average value representing trial performance.

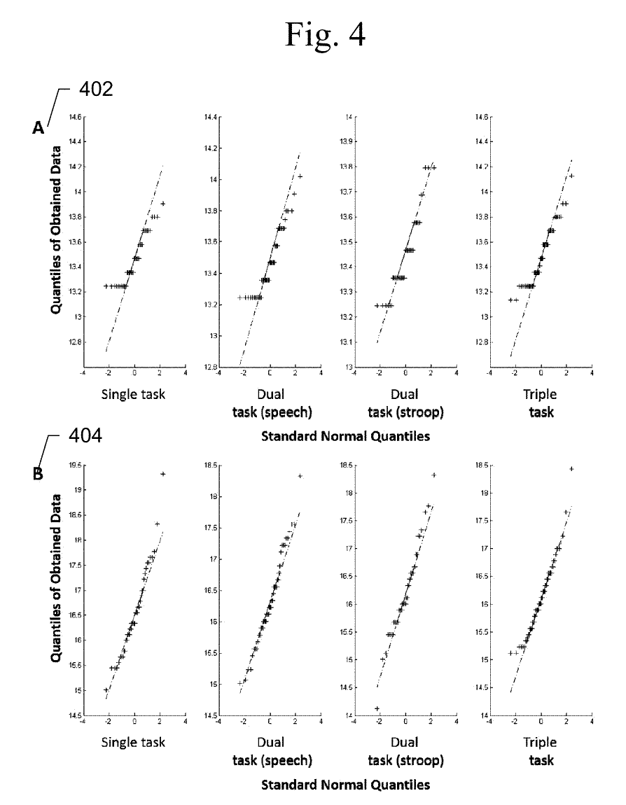

Statistical analysis may be applied to the results. For example, the Kolmogorov-Smirnov test may show that median frequency (fₘ) data is not normally distributed (p<0.01) for both the hand trajectories and accelerations. For example, the empirical cumulative distribution function of the collected data may be compared with the expected normal distribution, with a significant result indicating that the data is not normally distributed. Q-Q (quantile-quantile) plots may further confirm a non-Gaussian distribution with zero mean and unit variance. The Q-Q plots may be used to visually check for normality, as shown in FIG. 4. In the examples shown in FIG. 4, Q-Q plots showing the data across the four conditions for hand trajectories 402 and accelerations 404 are shown.

Boxplots may be used to visualize the data. A rank transformation procedure may be used in order to apply an analysis of variance on the data, with groups consisting of the 4 conditions (single task, dual with speech task, dual with Stroop task, and triple task). The ranked fₘ may be used as the dependent variable. Subsequently, non-parametric Kruskal-Wallis tests may be performed upon acceleration and position data to establish if any differences were present between conditions.

In embodiments, the performance outcome (fₘ) may follow a less ordered function. In order to explore this, a Naïve Bayes approach may be applied on the task limits, for example, the single and triple tasks. The Bayesian probability estimator may use the predictors of hand trajectory fₘ and acceleration fₘ to classify between the single and triple task condition. A Kernel smoothing density estimator may be applied for each predictor, as it was previously indicated that the data did not follow normality (see FIG. 4) and thus the density may be estimated based on all the available data points. The prior probabilities may be estimated from the relative frequencies of the single and triple task condition. The input feature matrix (x) may include fₘ columns for the hand position and acceleration, with Cᵢ representing the two possible classes (i=1 for single task; i=2 for triple task), as described by Bayes' Rule below.

$$P ( {C}_{I} \vert x ) = \frac{P ( x \vert {C}_{I} ) P ( {C}_{I} )}{P ( x )} \qquad ( 10 )$$

The probability that an observation belongs to a certain class (posterior probabilities) may be estimated using the predictor space, which may be defined by instances on a 2D-grid. The posterior probability that a classification is Cᵢ for a given observation may be computed by multiplying the conditional joint density of the predictors for a certain class with the class prior probability distribution and dividing it all by the joint density of the predictors. In embodiments, a condition with an increased probability for lower fₘ may yield a lower functional performance.

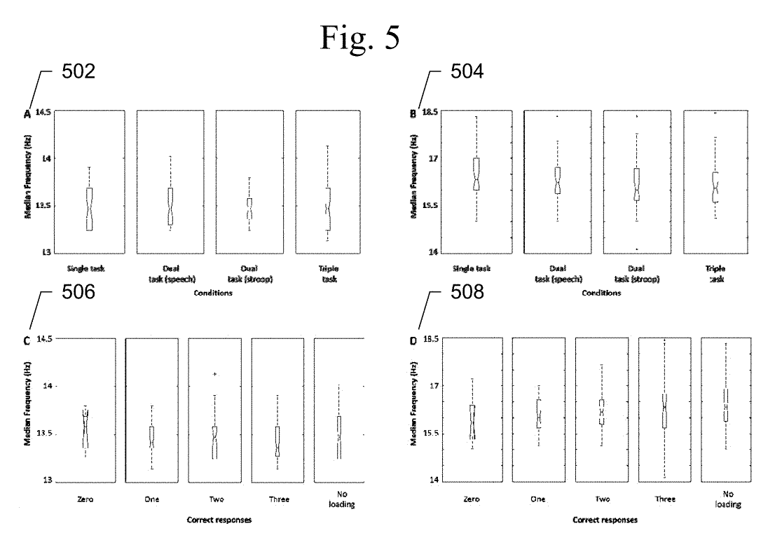

Boxplots may be used to visualize the hand trajectories and accelerations between conditions, an example of which is shown in FIG. 5. For example, ranked analysis of variance may show no significant difference between conditions for hand trajectory (F(3,195)=0.3170, p=0.81) and acceleration (F(3,195)=2.556, p=0.06). Likewise, the Kruskal-Wallis test may also find no significant differences for hand trajectory (H(3)=0.246, p=0.97) nor acceleration (H(3)=6.852, p=0.08). Further, the Bayesian probability estimator may show no clear task distinction based on the hand trajectory data. However, tasks may be differentiated based on the acceleration fₘ values between the single and triple tasks, for example, as shown in FIG. 5. In this example, the data indicates a clear distinction in the obtained fₘ between single and triple task performance.

As shown in the example of FIG. 5, boxplots are shown of the median frequency across the four conditions for hand trajectories 502 and accelerations 504. Boxplots are shown of the median frequency for trajectories 506 and accelerations 508 labelled by the total number of correct responses given for each trial. Trials that did not contain any Stroop task may be labelled as “no loading. The median value is shown as the central red mark and the edges of the box representing the 25th and 75th percentiles. The whiskers represent the most extreme data points and crosses are used for outliners.

In this example, applying this model for (same dataset) prediction may generate a misclassification of 38%, with most of the misclassification occurring in the fₘ of acceleration region between 15.8 and 17.2 Hz covering the 0.4 to 0.6 range of task probability. This region contained 33% of all data points. Values outside this region may yield a relatively good probability for separating the two tasks across all subjects. In general, higher fₘ values for accelerations were found in the single task, while low values more likely indicated subjects performing a triple task.

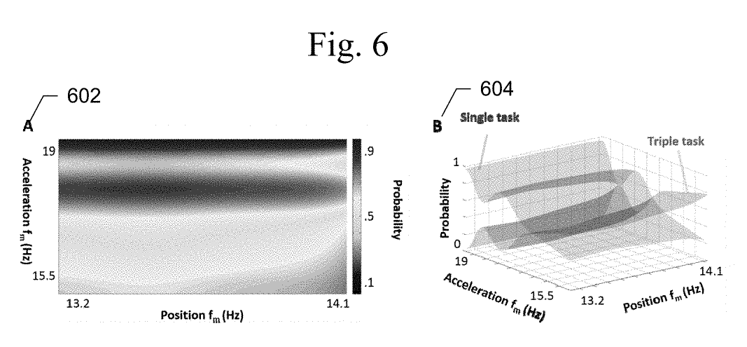

In this example, results showed no difference in hand trajectories between the conditions when traditional statistical methods such as the analysis of variance and Kruskal-Wallis test were used. However, visualization analysis of variance and the Kruskal-Wallis test indicated a clear trend towards lower fₘ for multitasking when the acceleration data was explored. The Bayesian probability estimator showed that differences existed in the probability estimates between the extremes (single and triple tasking), as shown in FIG. 6. Examples of visualisations of the estimated probability distribution between single and triple tasks are shown in FIG. 6. For example, at 602, a probability distribution between single and triple tasks is shown as a heat map, given the features of fₘ for position and acceleration. At 604, the same probability distribution between single and triple tasks is shown as in 602, but plotted in 3D for visualisation purposes. In this plot, a clear differentiation between the single task and the triple task is shown.

This differentiation between the single and triple task was also observed when the number of prepared sandwiches were counted. Participants completed fewer sandwiches when they were multitasking. This suggests that subjects may become “slower” both in fₘ accelerations, as well as in overall functional performance, when they are requested to multitask. The motor differences appear to be too small to be subjectively perceived as a decline in performance by subjects, but they become apparent by applying a simple Naïve Bayes model.

Our human perception bias often exists in quantifying our own performance and this bias is also found in caretakers assessing activities of daily living in those who suffer from a decline in cognitive abilities. A more objective approach to unobtrusively track function may therefore benefit both patients and clinical professionals. This kind of technology may especially impact those older adults who are living alone and the change attitude towards technologies can positively influence the uptake of these devices. It is important to consider that the activities described herein are very natural and intuitive.

Näive Bayes models may be useful for estimating general probability within real-time domains making it a suitable model for real-world tracking. Naïve Bayes models may also provide a computationally inexpensive method for differentiating between tasks and may be relatively easy to implement.

Although, real-world interaction is noisier, more heterogeneous and less repeatable than the induced Stroop task, the induced task does reflect the domain of interest. This makes it easier to robustly monitor and assess any potential changes. It would also indicate that cognitive load effects in the case of human-machine interfaces may be investigated in order to make them more ecologically valid.

This example shows that even simple everyday tasks performed by healthy individuals may be affected by multitasking for certain individuals. The potential to monitor this with an unobtrusive wearable sensor may be useful in relevant patient populations, such as Parkinson's disease (PD). Parkinson's disease (PD) is a progressive neurodegenerative disorder that affects the central nervous system and is primarily found in patients over 50 years of age. Symptoms include difficulty with motor skills such as walking and writing, as well as uncontrollable shaking (tremor), and general lethargy. These symptoms are caused by the death of neurons in the midbrain that control movement by generating dopamine, a neurotransmitter that modulates neural pathways and allows for smooth, controlled movement. In later stages of the disease, patients may experience trouble with emotional control and dementia. Studies have shown that early movement impairments and cognitive deficits can provide insight into the underlying neurodegenerative processes. In the case of Parkinson's disease, changes in physical movement typically precede changes in language and behaviour. Measurements of movement may therefore be particularly valuable as indicators of the earliest stages of neural dysfunction. In addition, impairments in PD may be exacerbated under simple dual-task conditions requiring the simultaneous performance of cognitive or motor tasks when compared to healthy controls. This provides further evidence that the described methods of monitoring activities of daily living under a range of conditions may predict changes at the executive level.

An increased probability of finding low median frequencies (fₘ) for wrist accelerations may be found during complex multitasking compared a single activity. It shows that even in healthy individuals who are performing everyday tasks, changes may arise in motor performance due to multitasking. Differentiation based on probability may occur at the extreme ends of the recorded values, while overlap exists within the midrange. Certain patient populations may show even more pronounced differences in motor performance during multitasking. The present systems and methods may provide the capability to measure this with a wearable sensor.

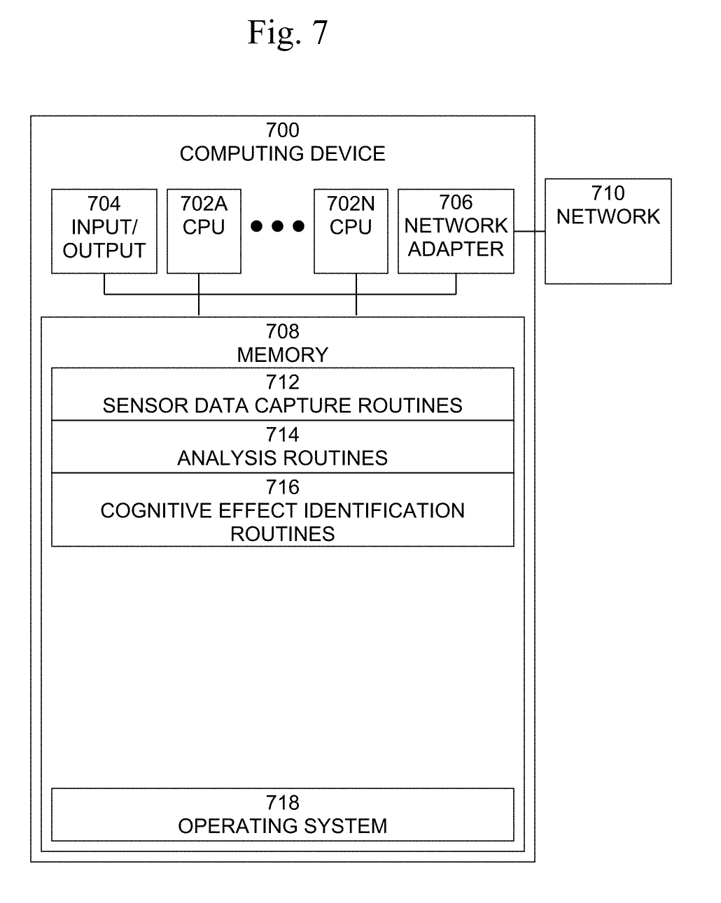

An exemplary block diagram of a computer system 702, in which processes involved in the embodiments described herein may be implemented, is shown in FIG. 7. Computer system 702 may be implemented using one or more programmed general-purpose computer systems, such as embedded processors, systems on a chip, personal computers, workstations, server systems, and minicomputers or mainframe computers, or in distributed, networked computing environments. Computer system 702 may include one or more processors (CPUs) 702A-702N, input/output circuitry 704, network adapter 706, and memory 708. CPUs 702A-702N execute program instructions in order to carry out the functions of the present communications systems and methods. Typically, CPUs 702A-702N are one or more microprocessors, such as an INTEL CORE® processor. FIG. 7 illustrates an embodiment in which computer system 702 is implemented as a single multi-processor computer system, in which multiple processors 702A-702N share system resources, such as memory 708, input/output circuitry 704, and network adapter 706. However, the present communications systems and methods also include embodiments in which computer system 702 is implemented as a plurality of networked computer systems, which may be single-processor computer systems, multi-processor computer systems, or a mix thereof.

Input/output circuitry 704 provides the capability to input data to, or output data from, computer system 702. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 706 interfaces device 700 with a network 710. Network 710 may be any public or proprietary LAN or WAN, including, but not limited to the Internet.

Memory 708 stores program instructions that are executed by, and data that are used and processed by, CPU 702 to perform the functions of computer system 702. Memory 708 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 708 may vary depending upon the function that computer system 702 is programmed to perform. In the example shown in FIG. 7, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In the example shown in FIG. 7, memory 708 may include sensor data capture routines 712, analysis routines 714, cognitive effect identification routines 716, and operating system 724. Sensor data capture routines 712 may include software routines to capture data from sensors, such as the wearable sensor shown in FIG. 1. Analysis routines 714 may include software routines to analyze the captured data to prepare it for identification of cognitive effect. Cognitive effect identification routines 716 may include software routines to identify cognitive effects, as described above. Operating system 720 may provide overall system functionality.

As shown in FIG. 7, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A computer-implemented method for determining effects of cognitive loading on a person comprising:
receiving data from at least one physical movement sensor attached to a person, the data recorded while the person is repeatedly performing a physical task and while the person is under cognitive loading during at least some of the performances of the task;
determining physical movements of the person in response to the cognitive loading from the received data for each of the performances of the task; and
determining an effect of cognitive loading based on the physical movements of the person while performing the physical task without cognitive loading and while performing the physical task with cognitive loading.

2. The method of claim 1, wherein the physical task is a task of everyday living.

3. The method of claim 2, wherein the cognitive loading is a Stroop task.

4. The method of claim 3, wherein determining physical movements of the person comprises:
determining a power spectrum of the sensor data;
comparing a frequency at which the power spectrum has a maximum amplitude with an expected range of frequencies; and
determining that a physical movement has occurred when the frequency at which the power spectrum has a maximum amplitude is within the expected range of frequencies.

5. The method of claim 4, wherein determining physical movements of the person further comprises:
applying a continuous wavelet transform to the sensor data of a physical movement for which it was determined that the physical movement occurred;
generating a scalogram of wavelet coefficients of the continuous wavelet transform;
determining that a physical movement began based on the scalogram;
determining the response time based on the time a stimulus was given and the determined time that the physical movement began; and
identifying the physical movement as correct when the determined physical movement matches an expected physical movement.

6. The method of claim 5, wherein determining an effect of cognitive loading comprises:
determining differences in response times between the physical tasks performed without cognitive loading and the physical tasks performed with cognitive loading.

7. The method of claim 6, wherein determining differences in response times comprises using a Naïve Bayes probability estimator to distinguish between the physical tasks performed without cognitive loading and the physical tasks performed with cognitive loading.

8. A system for identifying determining effects of cognitive loading on a person comprising:
at least one physical movement sensor attached to a person and adapted to transmit data representing physical movements of at least a portion of the person; and
a computing system comprising a processor, memory accessible by the processor, and computer program instructions stored in the memory and executable by the processor, computing system adapted to perform:
receiving data from the at least one physical movement sensor attached to the person, the data recorded while the person is repeatedly performing a physical task and while the person is under cognitive loading during at least some of the performances of the task,
determining physical movements of the person in response to the cognitive loading from the received data for each of the performances of the task, and
determining an effect of cognitive loading based on the physical movements of the person while performing the physical task without cognitive loading and while performing the physical task with cognitive loading.

9. The system of claim 8, wherein the physical task is a task of everyday living.

10. The system of claim 9, wherein the cognitive loading is a Stroop task.

11. The system of claim 10, wherein determining physical movements of the person comprises:
determining a power spectrum of the sensor data;
comparing a frequency at which the power spectrum has a maximum amplitude with an expected range of frequencies; and
determining that a physical movement has occurred when the frequency at which the power spectrum has a maximum amplitude is within the expected range of frequencies.

12. The system of claim 11, wherein determining physical movements of the person further comprises:
applying a continuous wavelet transform to the sensor data of a physical movement for which it was determined that the physical movement occurred;
generating a scalogram of wavelet coefficients of the continuous wavelet transform;
determining that a physical movement began based on the scalogram;
determining the response time based on the time a stimulus was given and the determined time that the physical movement began; and
identifying the physical movement as correct when the determined physical movement matches an expected physical movement.

13. The system of claim 12, wherein determining an effect of cognitive loading comprises:
determining differences in response times between the physical tasks performed without cognitive loading and the physical tasks performed with cognitive loading.

14. The system of claim 13, wherein determining differences in response times comprises using a Naïve Bayes probability estimator to distinguish between the physical tasks performed without cognitive loading and the physical tasks performed with cognitive loading.

15. A computer program product for determining effects of cognitive loading on a person, the computer program product comprising a non-transitory computer readable storage having program instructions embodied therewith, the program instructions executable by a computer, to cause the computer to perform a method comprising:
receiving data from at least one physical movement sensor attached to a person, the data recorded while the person is repeatedly performing a physical task and while the person is under cognitive loading during at least some of the performances of the task;
determining physical movements of the person in response to the cognitive loading from the received data for each of the performances of the task; and
determining an effect of cognitive loading based on the physical movements of the person while performing the physical task without cognitive loading and while performing the physical task with cognitive loading.

16. The computer program product of claim 15, wherein the physical task is a task of everyday living.

17. The computer program product of claim 16, wherein the cognitive loading is a Stroop task.

18. The computer program product of claim 17, wherein determining physical movements of the person comprises:
determining a power spectrum of the sensor data;
comparing a frequency at which the power spectrum has a maximum amplitude with an expected range of frequencies; and
determining that a physical movement has occurred when the frequency at which the power spectrum has a maximum amplitude is within the expected range of frequencies.

19. The computer program product of claim 18, wherein determining physical movements of the person further comprises:
applying a continuous wavelet transform to the sensor data of a physical movement for which it was determined that the physical movement occurred;
generating a scalogram of wavelet coefficients of the continuous wavelet transform;
determining that a physical movement began based on the scalogram;
determining the response time based on the time a stimulus was given and the determined time that the physical movement began; and
identifying the physical movement as correct when the determined physical movement matches an expected physical movement.

20. The computer program product of claim 19, wherein determining an effect of cognitive loading comprises:
determining differences in response times between the physical tasks performed without cognitive loading and the physical tasks performed with cognitive loading.

21. The computer program product of claim 20, wherein determining differences in response times comprises using a Naïve Bayes probability estimator to distinguish between the physical tasks performed without cognitive loading and the physical tasks performed with cognitive loading.
